(19) United States
(12) Patent Application Publication
(10) Pub. No.: US 2004/0162554 A1
(43) Pub. Date: Aug. 19, 2004

(54) Systems and methods for cutting tissue

(75) Inventors: Roberta Lee, Redwood City, CA (US); Samuel E. Zuckswert, San Jose, CA (US); Stanley R. Conston, San Carlos, CA (US); Ronald Yamamoto, San Francisco, CA (US); Huddee Jacob Ho, San Jose, CA (US)

Correspondence Address: Jung-hua Kuo, Attorney At Law, PO Box 3275, Los Altos, CA, 94024, US

(73) Assignee: MANOA MEDICAL, INC., A DELAWARE CORPORATION, Redwood City, CA

(21) Appl. No.: 10/744,462

(22) Filed: Dec. 22, 2003

Related U.S. Application Data
(60) Provisional application No. 60/435,972, filed on Dec. 20, 2002.

(51) Int. Cl. A61B 18/14

(52) U.S. Cl. 606/45

(57) ABSTRACT

Devices and methods for cutting of soft tissue using radio frequency and suitable for use in minimally invasive procedures are disclosed. The device generally includes an electrode housed in a housing, the electrode being flexible and/or extendable out of and/or retractable into the housing, and an insulating layer partially surrounding the electrode to expose at least a portion of the electrode to define at least one cutting area so as to focus energy from the energy source to the cutting area to facilitate initiation of a cut with the cutting area in contact with tissue. The cutting area(s) may extend and/or be aligned in a direction along a length of the electrode and may include a sharpened and/or serrated edge. The tissue cutting device may include an automated electrode oscillator coupled to the electrode and configured to oscillate the electrode along an axis or plane generally defined by the electrode.

### CROSS REFERENCE TO RELATED APPLICATIONS

This application claims priority to U.S. Provisional Patent Application Serial No. 60/435,972, entitled “Method for Cutting Tissue” and filed on Dec. 20, 2002, the entirety of which is incorporated by reference herein.

### BACKGROUND OF THE INVENTION

1. Field of the Invention

The present invention relates generally to cutting of tissue. More specifically, devices and methods for cutting of soft tissue using radio frequency and suitable for use in minimally invasive procedures are disclosed.

2. Description of Related Art

Minimally invasive surgical procedures have instigated a need for refinement in dividing soft tissue within a confined operative field. Standard surgical technique is often inadequate or not possible in a narrow space or with limited movement associated with minimally invasive procedures, one example being minimally invasive procedures in the breast. Dividing breast parenchyma during a minimally invasive procedure has most successfully been accomplished with a sharpened surface at the advancing edge of an oscillating cannula. Oscillating cannulae are fixed in diameter thereby limiting the ability of these type of cutting devices to excise specimens of variable sizes.

Radio frequency or electrosurgery is a common form of energy used to divide soft tissue in open surgical procedures. Radio frequency energized electrodes can be configured to different shapes and sizes. In addition, the electrodes may be bendable allowing for changes in size and/or shape during a procedure. Flexibility of the electrode improves the ability to cut around lesions of different sizes unlike the rigid cutting cannulae making the radio frequency energized electrode a desired device and method of cutting soft tissue during a minimally invasive procedure.

In open surgical procedures, monopolar radio frequency is most frequently used. An active electrode, often a “pencil” tip, performs the cutting procedure while a larger return electrode is placed elsewhere on the patient's skin. The active electrode is activated or energized in air. Once energized in air, the active electrode is manually positioned near the tissue to initiate the cutting process. Energizing the active electrode in air allows the open circuit voltage in the active electrode to rise causing the current density within the electrode to increase to a sufficient level to initiate a cut. If the active electrode is energized when it is already in contact with or embedded within the tissue, the current dissipates through the active electrode into the tissue causing a decrease in the voltage in the active electrode that is insufficient to initiate a cut. Initiation of the cut is delayed or the cut may not initiate at all. During the period of attempting to initiate a cut, the energized active electrode is in continual contact with a small area of tissue. This area of tissue often becomes charred and blackened (i.e. eschar) with the production of sparking, smoke, and a dangerous rise in temperature. Within the confines of a minimally invasive procedure, problems arise from the need to energize the active electrode when the active electrode is already inserted within the soft tissue prior to activation or is otherwise in contact with tissue.

Electrosurgical methods may also utilize bipolar configurations for the electrode, where the return electrode is configured in a position near the active electrode to create a zone of high current density for dividing tissue. Difficulties in initiating a cut using bipolar electrosurgical methods while in contact with tissue may also occur similar to monopolar electrosurgical methods.

One method of initiating the cut using radio frequency energy with the active electrode in contact with tissue is by using an electrosurgical generator with specific electrical features. One type of generator sends an initial burst of increased voltage to initiate the cut. Such increases in voltage attempt to overcome the problem of current dissipation into the tissue. The cut may be initiated faster than without the early burst of voltage but often not without increased smoke, sparking, and eschar formation. Delivering a surge of increased voltage to the tissue may cause disturbing muscle contractions and may cause increased pain. In addition to safety issues, such generators may not be available and are not practical. They are an additional capital expenditure and are often designed to be used with electrodes designed for safe delivery of such high voltages.

What is therefore needed are devices and methods of improving the initiation of a radio frequency energized cutting process when the active electrode is positioned within soft tissue at the beginning of a procedure or if interrupted, during any part of the procedure.

### SUMMARY OF THE INVENTION

Devices and methods for cutting of soft tissue using radio frequency and suitable for use in minimally invasive procedures are disclosed. It should be appreciated that the present invention can be implemented in numerous ways, including as a process, an apparatus, a system, a device, or a method. Several inventive embodiments of the present invention are described below.

The device for improved cutting of soft tissue generally includes an electrode energized by an external radio frequency energy source that facilitates the tissue cutting process and, in particular, that facilitates initiation of a cut within soft tissue. Depending on the requirements of the intended procedure, the electrode may be configured to a variety of shapes and/or sizes and may be fixed or variable in shape and/or size. The energy is generally focused at a predetermined cutting area(s) of the electrode by electrically insulating the noncutting area of the electrode. The electrode may also be moved repetitively back and forth along a path defined by the configuration of the electrode. Movement improves the efficiency of the cut and if intermittent cutting areas are appropriately configured, the movement exposes all tissue within the path of the cut to the intermittent cutting areas.

The tissue cutting device generally includes an electrode in communication with an energy source and housed in a housing, the electrode being flexible and/or extendable out of and/or retractable into the housing, and an insulating layer partially surrounding the electrode to expose at least a portion of the electrode to define at least one cutting area so as to focus energy from the energy source to the cutting area to facilitate initiation of a cut with the cutting area in contact with tissue. The cutting area(s) may extend and/or be aligned in a direction along a length of the electrode and may include a sharpened and/or serrated edge. In one example, the cutting area may be a protuberance defined by the electrode extending through an opening defined in the insulating layer.

The cutting area may be defined by an opening defined by the insulating layer and the tissue cutting device may further include a thermal insulator at least partially disposed in the opening defined by the insulating layer. The insulating layer may include segments spaced apart along a length of the electrode. The tissue cutting device may optionally include a protective layer covering the cutting area and disposed between the electrode and the insulating layer, the protective layer having electrical insulation properties less than that of the insulating layer. In one embodiment, the electrode may be a nonconductive material with a discrete conductive area corresponding to each cutting area. The discrete conductive area may be a conductive coating on a surface of the nonconductive material and/or a conductive element disposed in the nonconductive material.

The housing may be a probe such that the electrode is extendible and retractable out of and into the probe. The probe may include an insulating layer such that the electrode is extendible and retractable out of and into the probe. The tissue cutting device may also include an automated electrode oscillator coupled to the electrode and configured to move the electrode back and forth along an axis generally defined by a length of electrode. The tissue cutting device may also include a tissue collector, e.g., a wire mesh or a deformable bag, coupled to at least one of the probe and the electrode.

The electrode may be a loop electrode and the housing may be a probe coupled to the loop electrode for positioning and moving the loop electrode in the tissue. The probe may define at least one opening from which the loop electrode is extendible out of the probe to a predetermined and/or variable size and is retractable into the probe. An angle between a plane generally defined by the loop electrode and an axis defined along the length of the probe may be fixed or variable. The tissue cutting device may also include an automated electrode oscillator to move the loop electrode back and forth in a plane generally defined by the loop electrode. A distal end of the loop electrode may be housed within the probe and coupled to a spring housed in the probe such that the spring expands and compresses within the probe as a proximal end of the loop electrode extends from and retracts into the probe, respectively. The loop electrode may be configured to oscillate relative to the probe in a direction generally along a length of the probe, a direction generally orthogonal to the length of the probe, a rotational direction about an axis generally parallel to the length of the probe, a rotational direction about an axis generally orthogonal to the axis along the length of the probe, a plane generally defined by the electrode, and/or an axis generally defined by a length and/or width of the electrode.

As another example, the tissue cutting device may generally includes a housing, an electrode in communication with an energy source and housed in the housing, an insulating layer partially surrounding the electrode and configured to expose at least a portion of the electrode to define at least one cutting area, and an electrode oscillator coupled to the electrode and configured to oscillate the electrode. The electrode oscillator may oscillate the electrode relative to the housing in a direction generally along a length of the housing, a direction generally orthogonal to the length of the housing, a rotational direction about an axis generally parallel to the length of the housing, a rotational direction about an axis generally orthogonal to the axis along the length of the housing, a plane generally defined by the electrode, and/or an axis generally defined by a length and/or width of the electrode.

A method for cutting tissue generally includes exposing at least one cutting area of a cutting device to tissue, the cutting device including an electrode coupled to an energy source, an insulating layer partially surrounding the electrode and configured to expose at least a portion of the electrode to define the at least one cutting area, applying an energy from an external energy source to the electrode, the energy being generally focused in the at least one cutting area in contact with the tissue, and oscillating the at least one cutting area relative to the tissue using for example, a motor or a solenoid to cut the tissue. The oscillation may be, for example, at a frequency of between approximately 1 Hz and approximately 100 Hz and have a peak-to-peak distance of between approximately less than 1 mm to approximately 20 mm. The oscillation may be performed with a predetermined direction, a predetermined distance, and/or a predetermined frequency.

As another example, a method for cutting tissue may generally include exposing at least one cutting area of a cutting device to tissue, the cutting device including an electrode coupled to an energy source and housed in a housing, an insulating layer partially surrounding the electrode and configured to expose at least a portion of the electrode to define the at least one cutting area, the electrode being at least one of flexible, extendable out of the housing, and retractable into the housing and applying an energy from an external energy source to the electrode, the energy being generally focused in the at least one cutting area in contact with the tissue to cut the tissue.

These and other features and advantages of the present invention will be presented in more detail in the following detailed description and the accompanying figures which illustrate by way of example the principles of the invention.

### BRIEF DESCRIPTION OF THE DRAWINGS

The present invention will be readily understood by the following detailed description in conjunction with the accompanying drawings, wherein like reference numerals designate like structural elements.

FIGS. 1A-1D are perspective views illustrating various embodiments of electrically insulated electrodes.

FIGS. 2A and 2B are perspective views illustrating configurations of a modified electrode in relation to an outer insulating layer.

FIGS. 3A-3D are perspective views illustrating various embodiments of electrodes with a discontinuous insulating layer.

FIGS. 3E and 3F are a side and a top view, respectively, illustrating various embodiments of electrodes with an insulating layer and insulating segments.

FIGS. 4A-4D are perspective views illustrating movement of an exemplary electrode.

FIGS. 5A-5J illustrate movement of an exemplary electrode.

FIGS. 6A and 6B are a cross-sectional view and a side view, respectively, illustrating possible directions of movement of an exemplary electrode and a probe in combination.

FIGS. 7A and 7B illustrate possible directions of movement of a circle electrode.

FIG. 8 is a side view illustrating movement of a side loop electrode configured to actuate out of a generally linear slot on the side of the probe.

### DESCRIPTION OF SPECIFIC EMBODIMENTS

Devices and methods for cutting of soft tissue using radio frequency and suitable for use in minimally invasive procedures are disclosed. The following description is presented to enable any person skilled in the art to make and use the invention. Descriptions of specific embodiments and applications are provided only as examples and various modifications will be readily apparent to those skilled in the art. The general principles defined herein may be applied to other embodiments and applications without departing from the spirit and scope of the invention. Thus, the present invention is to be accorded the widest scope encompassing numerous alternatives, modifications and equivalents consistent with the principles and features disclosed herein. For purpose of clarity, details relating to technical material that is known in the technical fields related to the invention have not been described in detail so as not to unnecessarily obscure the present invention.

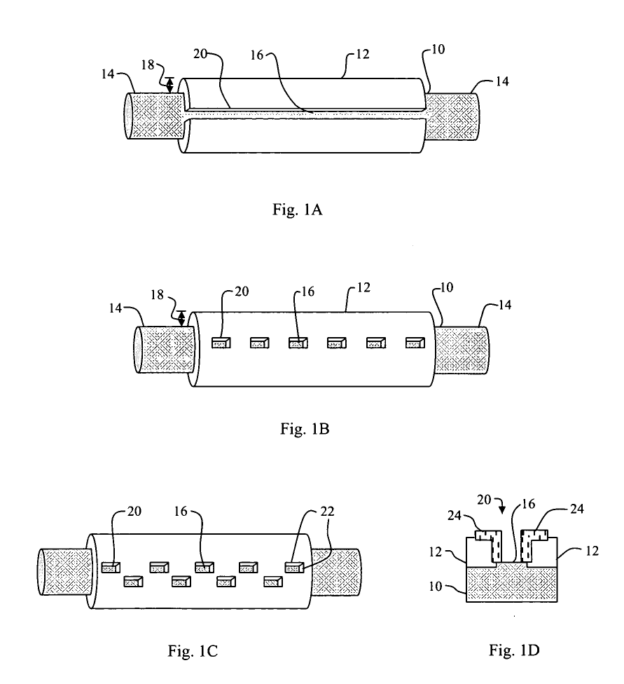

FIGS. 1A-C illustrate embodiments of a segment of an electrode 10 configured with an externally placed insulating layer 12 with a preferably high dielectric strength. The electrode 10 is in electrical communication with and energized by an external radio frequency generator (not shown). In the embodiment shown in FIG. 1A, the insulating layer 12 surrounds most of the electrode 10. For purposes of illustration, exposed ends 14 of the electrode 10 on either side of the insulating layer 12 are shown. In this exemplary segment of electrode 10, the insulating layer 12 includes a window 20 having a generally linear geometry and exposing a cutting area 16 on the underlying electrode 10. The cutting area 16 is defined by that part of the electrode 10 that is not covered by the insulating layer 12. In the exemplary embodiment, although the cutting area 16 shown is straight, the cutting area 16 may have regular or irregular bends and undulation. The electrode 10 may be made from a variety of materials, including but not limited to stainless steel, tungsten, other metals and alloys such as nickel titanium. The cross sectional area of the electrode 10 may be one of but not limited to a circle, oval, square, diamond, triangle, rectangle or other polygon. One or more edges of the electrode 10 may be sharpened or serrated. Preferably, the one or more edges that are sharpened or serrated are positioned in the cutting area 16.

The insulating layer 12 may be of a predetermined thickness 18 depending on the dielectric strength of the material used to construct the insulating layer 12. A material with a high dielectric strength will require less thickness 18 to electrically insulate the electrode 10 than a material with a low dielectric strength. In addition the thickness 18 may vary in different regions of the insulating layer 12, corresponding to different regions of the electrode 10 depending on the cutting properties required of the electrode 10. The insulating layer 12 may be constructed from any material with a desired dielectric strength such as but not limited to plastics, polymers (e.g. parylene, polytetrafluoroethylene, polyimide, polyetheretherketone, polydimethylsiloxanes), ceramics, and glass. Methods of coating the electrode 10 with the insulating layer 12 are well known to those skilled in the art and may include but are not limited to sputtering, plasma spraying, vacuum deposition, and mechanical securing of a sheath material such as heat shrink tubing. Methods of keeping the cutting area 16 of the electrode 10 from being covered by the insulating layer 12 are well known to those skilled in the art and may include but are not limited to masking the cutting area 16 prior to applying the insulating layer 12 and cutting away part of the insulating layer 12 using, for example, mechanical punches, laser machining techniques, and electrical discharge machining, after the insulating layer 12 has been applied. Depending on the configuration of the electrode 10, the insulating layer 12 may be stiff or flexible. In an alternative, the electrode 10 may be chemically treated (e.g. pickling) to create an oxide layer on the outer surface of the electrode 10. The oxide layer, depending on its thickness and dielectric strength, may act as an insulator. Windows in the oxide layer exposing the underlying cutting areas may be configured after the oxide layer is created by scraping away segments of the oxide layer or by masking segments of the oxide layer prior to treating the electrode 10. In a further alternative, ion implantation can be used in predetermined areas of the electrode surface. The ions may alter the electrical conductivity of the outer surface of the electrode to create cutting and insulating areas.

Preferably the insulating layer 12 is made of a material that is not affected or is minimally affected by high temperatures that can sometimes occur during activation and use of the electrode 10. When the electrode 10 is activated by an external radio frequency energy source (not shown), the insulating layer 12 minimizes current dissipation into the surrounding soft tissue thus allowing a more rapid increase of current density within the electrode 10 at lower voltages and improving the ability of the electrode 10 to initiate a cut. The cut initiates in the area of soft tissue that is in proximity to the cutting area 16 of the electrode 10. If activation of the electrode 10 is stopped for any reason during a procedure, the cut can resume without difficulty once the electrode 10 is re-activated. Other advantages of a partially insulated electrode are lower voltage requirements with improved safety, less charring of tissue and less build up of eschar on the electrode. A return electrode (not shown) to complete the radio frequency circuit may be a large dispersive electrode located elsewhere on the patient's skin as in a monopolar system. In an alternative, the return electrode may be located on or near the device as in a bipolar system.

In the embodiment shown in FIG. 1B, the insulating layer 12 surrounding the electrode 10 has multiple windows 20 defining multiple cutting areas 16. Preferably the multiple windows 20 are linearly aligned along a predetermined region of the electrode 10. In an alternative the multiple windows 20 may not be linearly aligned and may be arranged in a pattern as in the embodiment shown in FIG. 1C or randomly arranged (not shown). In some electrode configurations, it may be preferred to have at least a portion of the electrode window aligned such that the planar dimensions of the window are orthogonal or at one or more angles to a preferential direction of cutting. The multiple windows 20 may comprise any combination of number, shape, and/or size that provide the optimal cutting areas 16 for the intended procedure. For example, the windows 20 may be round, oval, square, rectangular, diamond and/or polygonal in shape. Edges 22 of the windows 20 may be sharp, rounded, angled or tapered. The distance between the windows 20 may be constant or varied. The thickness 18 of the insulating layer 12 generally defines the depth of the windows although the edges of the windows may be angled or tapered. FIG. 1D is a cross-sectional view of an exemplary window 20. One or more materials may be configured as a window insert 24 to fit within at least one window 20 to enhance cutting of soft tissue or provide safety features to the electrode 10 and insulating layer 12. For example, the window insert 24 may modify the shape of the window 20 or may improve current conductivity through the window 20. The window insert 24 may cover the edges of the insulating layer 12 as shown in FIG. 1D or may completely fill the window 20 (not shown). As an additional example, a window insert 24 may act as a thermal insulator protecting the insulating layer 12 at the edges of the window 20 from high temperatures that may result during the cutting process. The window insert 24 acting as a heat insulator may be made from a variety of materials (e.g. ceramics, glass, polymers) well known to those skilled in the art. One or more cutting areas 16 may contain a current concentrator such as one or more edges or one or more tips. The edges may extend into the electrode or may protrude from the electrode as ridges. The edges or tips may be sharpened.

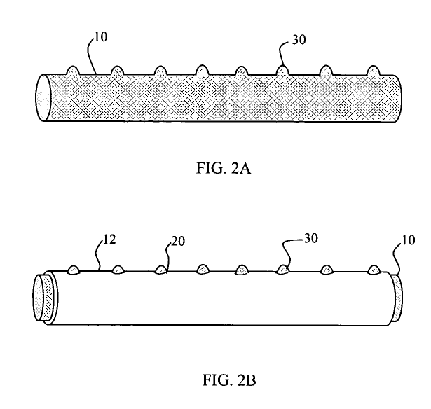

In the embodiment shown in FIGS. 2A-B, the electrode 10 is configured to have at least one protuberance 30 that is electrically conductive. The at least one protuberance 30 may be of variable size, shape, number, location, and/or distance apart from each other. Preferably the at least one protuberance 30 is linearly aligned on the electrode 10 but in the alternative, the at least one protuberance 30 may not be linearly aligned. The protuberance 30 and the electrode 10 may be configured as a single unit as illustrated in FIG. 2A but in an alternative (not shown), the protuberance 30 may be made of a separate fixture that is fastened to the electrode 10. In the embodiment illustrated in FIG. 2B, the electrode 10 has an insulating layer 12 that is thinner in thickness than the height of the protuberances 30 thereby exposing part of the protuberances 30 through windows 20 in the insulating layer 12. The exposed length of the protuberance 30 external to the insulating layer 12 is defined by the height of the protuberance 30 and the thickness of the insulating layer 12. The insulating layer 12 minimizes current dissipation into tissue and allows the voltage to increase in the protuberances 30 to initiate the cut when the electrode 10 is energized.

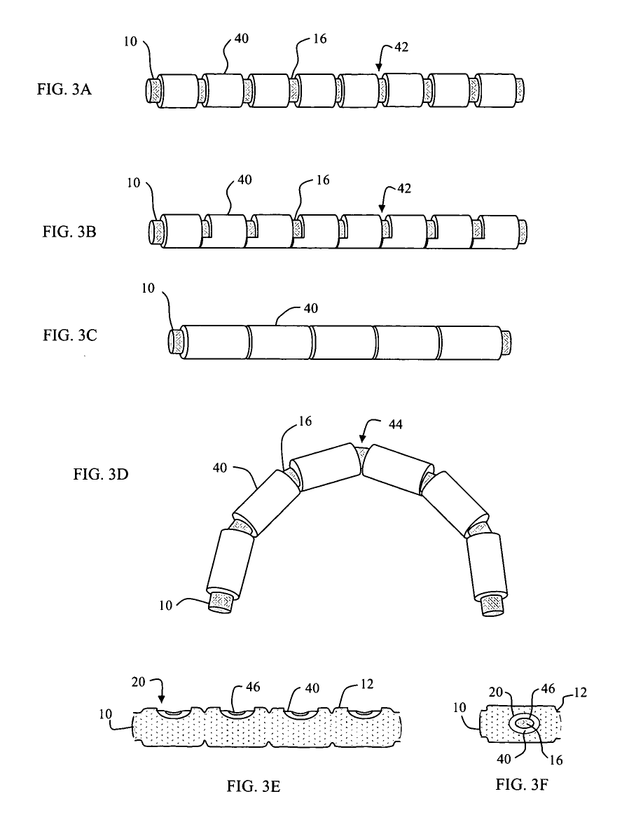

In the embodiments shown in FIGS. 3A-3D, multiple insulating segments 40 surround the electrode 10. In the embodiment shown in FIG. 3A, spaces 42 between the insulating segments 40 expose the underlying electrode 10 to the tissue thereby defining the cutting areas 16. The insulating segments 40 are configured to provide an optimal number, shape, and size of the cutting areas 16. In the embodiment shown in FIG. 3B, a part of each insulating segment 40 completely surrounds the electrode 10 while the remainder of the insulating segment 40 insulates only part of the electrode. In another embodiment shown in FIGS. 3C and 3D, the insulating segments 40 are configured to insulate an electrode 10 that can assume a bent or rounded shape. As shown in FIG. 3C, the insulating segments 40 cover the electrode 10 when the electrode 10 is in a linear configuration. As shown in FIG. 3D, when the electrode 10 is configured to a bent or loop shape, the insulating segments 40 that are generally rigid and linear in geometry do not cover the electrode 10 evenly. A gap 44 between each insulating segment 40 exposes the underlying electrode 10 thereby configuring the cutting areas 16. Cutting areas 16 are generally defined around the circumference of the electrode 10 in this exemplary embodiment. The thickness, length, and shape of the insulating segments 40 are predetermined to optimize the cutting ability of the electrode 10. Individual insulating segments 40 surrounding an electrode 10 may also be of varying thickness, length and shape (not shown). In an alternative, one or more conducting segments (not shown) may alternate with the insulating segments. The conducting segments conduct the radio frequency energy to adjacent tissue. The conducting segments may vary in size and shape and may contain a current concentrator.

In yet a further alternative, as illustrated in a side view in FIG. 3E, and a top view of a single insulating segment 40 in FIG. 3F, the insulating segments 40 have one or more openings 46 preferably on one side of the insulating segment 40. The insulating segments 40 are preferably oriented around the electrode 10 such that the one or more openings 46 are generally linearly aligned. The one or more openings 46 define the cutting areas 16. Covering the insulating segments 40 and the electrode 10 is an insulating layer 12. The insulating layer 12 contains one or more windows 20 that expose the underlying one or more openings 46. When the electrode 10 is activated with radio frequency energy from an external source (not shown), the insulating segments 40 and the insulating layer 12 prevent current dissipation into tissue. The current density is concentrated in the electrode 10, thereby causing the cutting areas 16 to initiate and continue a cut in the adjacent tissue. In addition, the insulating segments 40 act as a thermal insulator protecting the insulating layer 12 from high temperatures that can be reached when the electrode 10 is activated with radio frequency energy. The insulating layer 12 may also have lubricious qualities and enhance the ability of the insulating segments 40 and electrode 10 to move through channels within a probe (not shown) for extension and retraction of the electrode 10 in the tissue. The insulating layer 12 may also facilitate the prevention of eschar build up on the electrode 10.

The above embodiments illustrate cutting areas comprising portions of the electrode not covered with the insulating layer. In an alternative (not shown), the entire electrode or only the cutting areas may be coated with a protective layer that may have some electrically insulative properties but is able to conduct current to the tissue through capacitive coupling. The protective layer prevents eschar from accumulating in the cutting areas. The protective layer may be made from materials well known to those skilled in the art and may include but are not be limited to plastics, polymers, metal, ceramics, and glass. The thickness of the protective layer and the dielectric strength of the material used to configure the protective layer determine the ability of the protective layer to conduct the radio frequency energy. In another embodiment (not shown), the electrode may be made from a nonconductive material, with discrete electrode areas created on the surface with the use of conductive coatings or conductive elements placed in the core of the nonconductive material that are exposed to the surface in discrete locations.

Electrodes energized with radio frequency energy, either with or without at least a partial cover of insulation, may be provided with the additional energy of mechanical movement back and forth along the plane of the cut, the plane of the cut being defined as the generally orthogonal to the path or direction of the cut. The mechanical movement may facilitate the cutting process by allowing different areas of the electrode to contact different areas of the tissue during the cutting process. Furthermore, when the cutting area(s) is sharpened and/or serrated, the movement may further facilitate the cutting process through the mechanical cutting of tissue with the sharpened or serrated cutting area(s). The movement may also help prevent the tissue from adhering to the electrode. The movement may also facilitate in allowing the cutting areas of an electrode covered with interrupted (e.g., segmented) insulation to contact the entire plane of the cut.

Although the mechanical movement may be provided manually, preferably, the mechanical movement is provided by an energized automated mechanism (e.g. motor and/or solenoid) such as through a level or knob on a handle of the device. The energized automated mechanism may be an electrode oscillator coupled to the electrode and configured to move the electrode back and forth along an axis generally defined by a length of electrode. The direction of the movement may be defined by the shape of the electrode configuration. The distance and frequency of each direction of movement may be predetermined and/or may be variable depending on the type or types of tissue being cut. The movement is preferably an oscillating or repetitive movement in which the peak-to-peak distance of the movement may be a low range (e.g., less than 1 mm), a medium range (e.g., 1 mm to 5 mm), or a high range (e.g., 5 mm to 20 mm). In addition, the frequency of the movement may range from 1 Hz to 100 Hz. Variations in the distance and frequency may be controlled, for example, by one or more dials on the handle of the device.

In one embodiment, the electrode and insulating layer oscillate in unison. In another embodiment, the insulating layer acts as an insulating sleeve that oscillates back and forth over the electrode to expose different areas on the electrode to the tissue to act as cutting areas. After the electrode is configured to the desired shape and size, the electrode advances along the path of the cut while the insulating sleeve moves back and forth over the electrode. Preferably the insulating sleeve moves in a direction that follows the longitudinal axis of the electrode but the insulating sleeve may also be configured to move perpendicular and/or at an angle to the longitudinal axis of the electrode. The configuration of the electrode (e.g. shape, size, fixed or variable), the type of insulation and the parameters of movement are preferably optimized for cutting depending on the type of tissue and procedure to be performed.

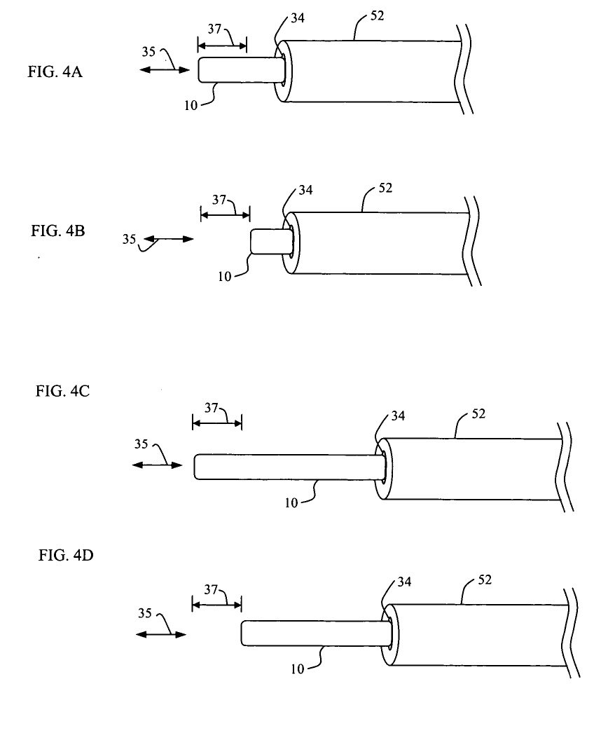

FIGS. 4-8 illustrate various configurations of the electrode 10 and the movement associated with each configuration. For ease of demonstration, the insulating layer(s) and/or insulating segments are not shown. It is noted that any suitable embodiments of the electrode 10, e.g., with and without the insulating layer(s) and/or insulating segments, may be employed in the configurations in FIGS. 4-8.

FIGS. 4A-4D illustrate an example of an electrode 10 mounted in a probe 52. As shown, the electrode 10 is illustrated without a separate insulating layer (the probe serves as an insulation layer) but the electrode 10 may be additionally configured with any suitable insulating layer such as one of the insulating layers described herein. The electrode 10 is configured to repetitively move out and in (i.e. back and forth) along an axis 35 defined by the length of the electrode 10. The movement distance 37 of each out and in movement may be predetermined or variable. The electrode 10 may also extend out from the probe 52 to a predetermined or variable length with the capability of moving back and forth along the axis 35 in the extended position as shown in FIGS. 4C and 4D. The electrode 10 may also be configured to move in one or more directions that are perpendicular or at an angle to the axis 35 or in a circular motion (not shown). With an electrode with discrete cutting and non-cutting areas, the motion may be configured such that the resultant path of each cutting area at least coincides with the path of each neighboring cutting area to result in a continuous path in which tissue is divided. Some overlap in the motion paths of neighboring cutting areas may be desired to accommodate tissue motion and ensure a continuous path of tissue dividing. The movement may also prevent tissue from sticking to the electrode which often results in blackened eschar and smoke. Accumulated eschar on the electrode decreases the cutting efficiency of the electrode. The movement may be manually controlled but in a preferable alternative, the movement is controlled by an energized mechanism that may be housed within the handle. The energized mechanism may be a motor powered by a battery, standard wall current, springs, air, hydraulic or any other mechanical mechanism. If the wall current is used, an AC adapter is preferably used to convert the current to DC, for example 12 volt DC. The direction, distance, and frequency of each back and forth movement may be fixed and predetermined or in an alternative, the direction, distance, and frequency may be varied, for example, by adjusting dials on the handle. In an alternative, the energized movement predetermined or variable may be derived from one or more solenoids. In the exemplary embodiment, the electrode is configured in the shape of a flat blade, as illustrated in FIGS. 4A-4D. The electrode may also be configured in one of many alternatives (not shown), including but not limited to a wire, a hook, a partial circle, a circle, square, rectangle, polygon, oval, and triangle. The electrode may be an irregular shape or have irregular features. Part or all of the electrode may be manually or mechanically bendable to achieve a desired configuration. Part or all of the electrode may have an insulating layer that is continuous or discontinuous.

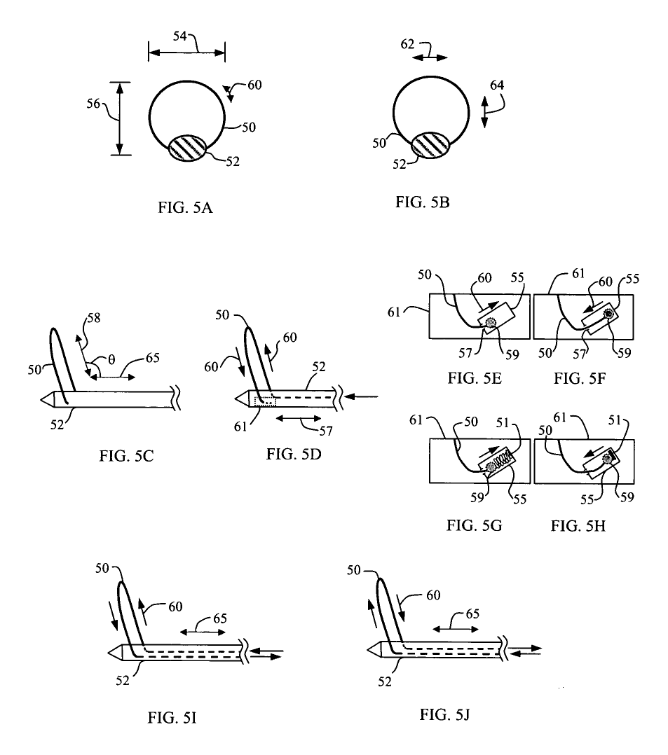

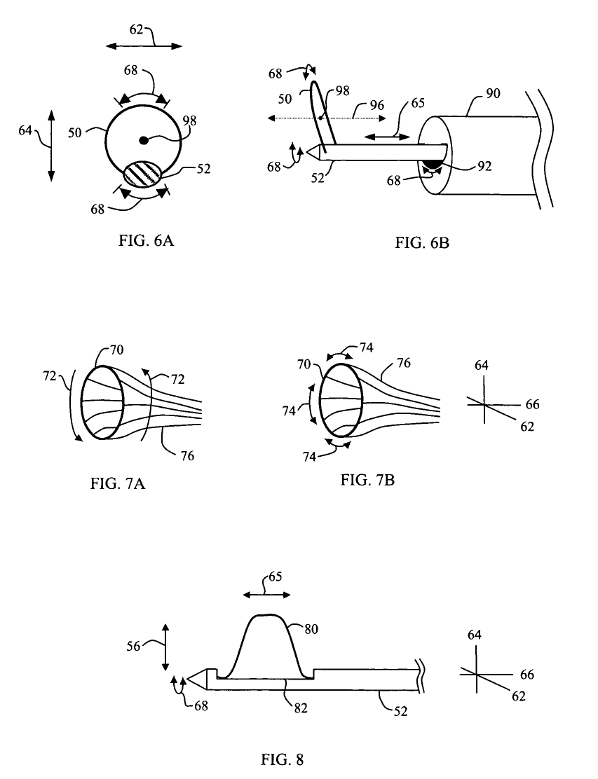

FIGS. 5A-5J and FIGS. 6A and 6B illustrate exemplary embodiments of the electrode 10 in the shape of a loop electrode 50. In FIGS. 5A-5J, both ends of the loop electrode 50 are housed within a probe 52 as shown in cross-sectional views in FIGS. 5A and 5B. The loop electrode 50 defines a horizontal dimension 54 and a vertical dimension 56. The horizontal dimension 54 and the vertical dimension 56 define a shape of the loop electrode 50. In the example illustrated in FIGS. 5A and 5B, the shape is one of a partial circle. The horizontal dimension 54 may be fixed in a predetermined length or may be changeable by controlling an exit angle (not shown) of one or both ends of the loop electrode 50 as it exits the probe 52. The vertical dimension 56 of the loop electrode 50 may be fixed at a predetermined length or changeable by actuating the loop electrode 50 to extend out of or retract into the probe 52. In addition, as illustrated in FIGS. 5C, an angle 0 defined by the relationship between a plane 58 defined by the loop electrode 50 and a probe axis 65 defined by the probe 52 may be fixed or changeable. In the embodiment shown in FIG. 5A, movement of the loop electrode 50 along a path 60, generally defined by the curvature of the loop electrode 50, can be performed manually or preferably by an energized mechanism (not shown) which causes the loop electrode 50 to move back and forth along the path 60. Preferably the movement along the path 60 is started before the electrode 10 is energized and continues as the electrode 10 is advanced or retracted to cut the tissue. Alternative movements shown in FIG. 5B are along direction 62 and/or along direction 64. In a further alternative, as shown in FIG. 5C, the movement may be along the probe axis 65.

FIGS. 5D-5J illustrate examples of the movement of the loop electrode 50. In FIGS. 5I and 5J, both ends of the loop electrode 50 are located in a proximal region of the probe 52 or handle (not shown) and are attached to an energized mechanism (not shown) and moved back and forth. FIG. 5I illustrates movement in one direction while FIG. 5J illustrates movement in the opposite direction. Movement of the proximal end of the loop electrode 50 back and forth along probe axis 65 causes a segment of the loop electrode 50 that is outside of the probe 52 to move back and forth in the path 60. FIGS. 5E-5H illustrate various embodiments of section 61 as shown in dashed in FIG. 5D in more detail. In FIG. 5E, a distal end 59 of the loop electrode 50 is housed in a compartment 55 located near or at the distal end of the probe 52. A proximal end (not shown) of the loop electrode 50 is located in the proximal region of the probe 52 or handle (not shown) and is attached to an energized mechanism (not shown). The distal end 59 of the loop electrode 50 moves back and forth in the compartment 55. As illustrated in FIGS. 5E and 5F, the distal end 59 may be configured to be larger than an opening 57 in the compartment 55 through which the loop electrode 50 passes such that the distal end 59 remains within the compartment 55 when the loop electrode 50 moves back and forth along direction 60. In addition, as illustrated in FIGS. 5G and 5H, a spring 51 may be attached to the distal end 59. The spring 51 compresses (FIG. 5H) and relaxes (FIG. 5G) within the compartment 55 as the loop electrode 50 is moved back and forth along direction 60.

In the embodiment shown in FIGS. 6A and 6B, the probe 52 and loop electrode 50 move more or less in unison back and forth along a predetermined path 68. FIG. 6A is a cross-sectional view of the probe 52. The direction of the path 68 is preferably defined by the curvature of the loop electrode 50. As illustrated in a side view of the embodiment in FIG. 6B, a slot 92 in a distal end of the handle 90 allows the probe 52 and the loop electrode 50 to move back and forth in a direction defined by the path 68. The path 68 is preferably equidistant from a movement axis 96 that is parallel to the probe axis 65 and intersects a loop center 98 of the loop electrode 50. As the loop electrode 50 changes in height, the distance of the loop center 98 relative to the probe 52 will change. Preferably the path 68 is automatically or manually adjustable such that the movement axis 96 continues to intersect the loop center 98 with variable height of the loop electrode 50. The distance and frequency of movement along the path 68 may be fixed and predetermined or one or both may be variable. The probe 52 and loop electrode 50 may also move as a single unit in at least one of the direction 62, the direction 64, as illustrated in FIG. 6A, and along the probe axis 65, as illustrated in FIG. 6B.

FIGS. 7A and 7B illustrate exemplary embodiments of the electrode 10 in the form of a generally circular electrode 70. A tissue collector such as a wire mesh 76 or a deformable bag (not shown) may or may not be attached to the circle electrode 70. The circle electrode 70 may be configured to rotate in a circular path 72 and/or may be configured to move back and forth in a curved path 74. The circle electrode 70 may be configured to move back and forth in at least one of the direction 62, the direction 64, and the direction 66, and/or at any angle relative to the direction 62, the direction 64, and the direction 66.

FIG. 8 illustrates an exemplary embodiment of the electrode 10 in the form of a side loop electrode 80 that is configured to actuate out of a generally linear slot 82 on the side of the probe 52. The side loop electrode 80 is configured to move back and forth along the probe axis 65 defined by the probe 52. An energized mechanism (not shown) is housed in the handle (not shown) and is attached to the proximal end of the side loop electrode 80. The distal tip (not shown) of the side loop electrode 80 is contained within a compartment (not shown) in the distal end of the probe 52. The distal tip of the side loop electrode 80 may be configured to be larger than an opening in the compartment through which the side loop electrode 80 passes. The compartment is of sufficient length to allow the distal tip of the side loop electrode 80 to traverse a distance approximately equal to the distance of each direction of movement generated by the energized mechanism. In an alternative, a spring may be attached to the distal tip. The spring is housed within the compartment and compresses and expands with each back and forth movement of the side loop electrode. In another alternative, the side loop electrode 80 may move back and forth in at least one of the direction 62, the direction 64, and the direction 66, and/or at any angle relative to direction 62, the direction 64, and the direction 66. In yet a further alternative, the side loop electrode 80 and the probe 52 may be configured to move in unison in any of the above described directions.

In an alternative, tissue cutting may be accomplished with the electrode 10 containing a sharpened or serrated edge in combination with movement of the electrode which may be sufficient to cut soft tissue without the addition of energy (e.g. radio frequency).

A tissue penetrator such as a cannula may be attached to one of the electrode and the probe. The tissue penetrator may be a sharpened edge or tip and/or may be energized, for example with radio frequency energy. In a further alternative, a tissue marking mechanism and/or a tissue collector may be attached to a least one of the electrode and the probe. For example, a tissue collection bag may be attached to part of the insulating layer covering the electrode. As the external surface of the insulating layer will not reach the high temperatures associated with radio frequency cutting, a tissue collection bag will be not be in danger of melting or dissolving at the site of attachment.

While the exemplary embodiments of the present invention are described and illustrated herein, it will be appreciated that they are merely illustrative and that modifications can be made to these embodiments without departing from the spirit and scope of the invention. Thus, the scope of the invention is intended to be defined only in terms of the following claims as may be amended, with each claim being expressly incorporated into this Description of Specific Embodiments as an embodiment of the invention.

What is claimed is:

1. A tissue cutting device, comprising:
a housing;
an electrode in communication with an energy source, the electrode being housed by the housing and configured to be at least one of flexible, extendable out of the housing, and retractable into the housing;
at least one cutting area comprising an exposed portion of the electrode, the cutting area being configured to generally focus energy from the energy source to the at least one cutting area to facilitate initiation of a cut with the at least one cutting area in contact with tissue; and
an insulating layer partially surrounding the electrode and configured to expose at least a portion of the electrode to define the at least one cutting area.

2. The tissue cutting device of claim 1, wherein the cutting area includes a cutting area extending in a direction along a length of the electrode.

3. The tissue cutting device of claim 1, comprising a plurality of cutting areas, the cutting areas generally aligned in a direction along a length of the electrode.

4. The tissue cutting device of claim 1, wherein the at least one cutting area includes at least one of a sharpened edge and a serrated edge.

5. The tissue cutting device of claim 1, wherein the cutting area is a protuberance defined by the electrode, the protuberance extending through an opening defined in the insulating layer.

6. The tissue cutting device of claim 1, wherein the at least one cutting area includes a plurality of the protuberances generally aligned in a direction along a length of the electrode.

7. The tissue cutting device of claim 1, wherein the at least one cutting area is defined by an opening defined by the insulating layer, further comprising a thermal insulator at least partially disposed in the opening defined by the insulating layer.

8. The tissue cutting device of claim 1, wherein the insulating layer includes a plurality of segments disposed along a length of the electrode, the segments being spaced apart relative to each other to expose at least a portion of the electrode to define the at least one cutting area.

9. The tissue cutting device of claim 1, wherein the insulating layer includes a plurality of segments disposed along a length of the electrode, each segment defining an opening to expose the electrode to define the at least one cutting area, the openings defined by the segments being generally aligned in a direction along a length of the electrode.

10. The tissue cutting device of claim 1, wherein the insulating layer comprises a material selected from the group consisting of plastic, polymer, parylene, polytetrafluoroethylene, polyimide, polyetheretherketone, polydimethylsiloxanes, ceramics, and glass.

11. The tissue cutting device of claim 1, further comprising a protective layer disposed between the electrode and the insulating layer, the protective layer having electrical insulation properties less than that of the insulating layer, the protective layer covering the at least one cutting area.

12. The tissue cutting device of claim 1, wherein the electrode comprises a nonconductive material and at least one discrete conductive area corresponding to the at least one cutting area, the conductive area being selected from the group consisting of a conductive coating on a surface of the nonconductive material and a conductive element disposed in the nonconductive material.

13. The tissue cutting device of claim 1, wherein the housing is a probe, the electrode being extendible and retractable out of and into the probe.

14. The tissue cutting device of claim 13, wherein the probe comprises the insulating layer, the electrode being extendible and retractable out of and into the probe.

15. The tissue cutting device of claim 13, further comprising an automated electrode oscillator coupled to the electrode and configured to oscillate the electrode back and forth along an axis generally defined by one of a length of the electrode and a width of the electrode.

16. The tissue cutting device of claim 1, wherein the electrode is a loop electrode and wherein the housing is a probe coupled to the loop electrode for positioning and moving the loop electrode in the tissue.

17. The tissue cutting device of claim 16, wherein the probe defines at least one opening from which the loop electrode is extendible out of the probe to at least one of a predetermined and variable size and is retractable into the probe.

18. The tissue cutting device of claim 16, wherein an angle between a plane generally defined by the loop electrode and an axis defined along the length of the probe is one of fixed and variable.

19. The tissue cutting device of claim 16, further comprising an automated electrode oscillator coupled to the loop electrode and configured to move the loop electrode back and forth in a plane generally defined by the loop electrode.

20. The tissue cutting device of claim 16, wherein the loop electrode includes a proximal end and a distal end and wherein the distal end is housed within the probe, the tissue cutting device further comprising a spring housed in the probe and coupled to the distal end of the loop electrode such that the spring expands and compresses within the probe as the proximal end of the loop electrode extends from and retracts into the probe, respectively.

21. The tissue cutting device of claim 16, wherein the loop electrode is configured to be oscillated relative to the probe in at least one of a direction generally along a length of the probe, a direction generally orthogonal to the length of the probe, a rotational direction about an axis generally parallel to the length of the probe, a rotational direction about an axis generally orthogonal to the axis along the length of the probe, a plane generally defined by the electrode, an axis generally defined by a length of the electrode, and an axis generally defined by a width of the electrode.

22. The tissue cutting device of claim 16, wherein the loop electrode extends from one of a distal end of the probe and a side of the probe adjacent to the distal end of the probe.

23. The tissue cutting device of claim 16, further comprising a tissue collector coupled to the probe, the tissue collector being selected from the group consisting of a wire mesh and a deformable bag.

24. The tissue cutting device of claim 23, wherein the tissue collector is further coupled to the loop electrode.

25. A tissue cutting device, comprising:
a housing;
an electrode in communication with an energy source, the electrode being housed in the housing;
an insulating layer partially surrounding the electrode and configured to expose at least a portion of the electrode to define at least one cutting area; and
an electrode oscillator coupled to the electrode and configured to oscillate the electrode relative to the housing in at least one of a direction generally along a length of the housing, a direction generally orthogonal to the length of the housing, a rotational direction about an axis generally parallel to the length of the housing, a rotational direction about an axis generally orthogonal to the axis along the length of the housing, a plane generally defined by the electrode, an axis generally defined by a length of the electrode, and an axis generally defined by a width of the electrode.

26. The tissue cutting device of claim 25, wherein the electrode oscillator oscillates the electrode at a frequency of between approximately 1 Hz and approximately 100 Hz.

27. The tissue cutting device of claim 25, wherein the electrode oscillator oscillates the electrode at a peak-to-peak distance of between approximately less than 1 mm to approximately 20 mm.

28. A method for cutting tissue, comprising the steps of:
exposing at least one cutting area of a cutting device to tissue, the cutting device including an electrode coupled to an energy source, an insulating layer partially surrounding the electrode and configured to expose at least a portion of the electrode to define the at least one cutting area;
applying an energy from an external energy source to the electrode, the energy being generally focused in the at least one cutting area in contact with the tissue; and
oscillating the at least one cutting area relative to the tissue to cut the tissue.

29. The method for cutting tissue of claim 28, wherein the electrode is housed in a housing and wherein the oscillating is performed by extending and retracting the electrode into and out of the housing.

30. The method for cutting tissue of claim 28, wherein the oscillating includes oscillating the at least one cutting area relative to a probe housing the electrode, the oscillating being performed by an automated electrode oscillator coupled to the electrode.

31. The method for cutting tissue of claim 30, wherein the automated electrode oscillator oscillates the electrode at a frequency of between approximately 1 Hz and approximately 100 Hz.

32. The method for cutting tissue of claim 30, wherein the automated electrode oscillator oscillates the electrode at a peak-to-peak distance of between approximately less than 1 mm to approximately 20 mm.

33. The method for cutting tissue of claim 30, wherein the automated electrode oscillator includes at least one of a motor and solenoid.

34. The method for cutting tissue of claim 28, wherein the oscillating is performed with at least one of a predetermined direction, a predetermined distance, and a predetermined frequency.

35. The method for cutting tissue of claim 28, wherein the oscillating is oscillating the electrode into and out of the probe.

36. A method for cutting tissue, comprising the steps of:
exposing at least one cutting area of a cutting device to tissue, the cutting device including an electrode coupled to an energy source and housed in a housing, an insulating layer partially surrounding the electrode and configured to expose at least a portion of the electrode to define the at least one cutting area, the electrode being at least one of flexible, extendable out of the housing, and retractable into the housing; and
applying an energy from an external energy source to the electrode, the energy being generally focused in the at least one cutting area in contact with the tissue to cut the tissue.

37. The method for cutting tissue of claim 36, further comprising oscillating the at least one cutting area relative to the tissue to further facilitate cutting of the tissue.

38. The method for cutting tissue of claim 36, wherein the cutting area includes a cutting area extending in a direction along a length of the electrode.

39. The method for cutting tissue of claim 36, wherein the at least one cutting area includes a plurality of cutting areas generally aligned in a direction along a length of the electrode.

40. The method for cutting tissue of claim 36, wherein the at least one cutting area includes at least one of a sharpened edge and a serrated edge.

41. The method for cutting tissue of claim 36, wherein the insulating layer includes a plurality of segments disposed along a length of the electrode, the segments being spaced apart relative to each other to expose at least a portion of the electrode to define the at least one cutting area.

42. The method for cutting tissue of claim 36, further comprising the step of flexing the electrode to expose the at least one cutting area to the tissue to be cut.
